(19) United States
(12) Patent Application Publication
(10) Pub. No.: US 2016/0213400 A1
(43) Pub. Date: Jul. 28, 2016

(54) HAIR TRANSPLANTER

(71) Applicant: Kun OC, Seoul (KR)

(72) Inventor: Kun OC, Seoul (KR)

(21) Appl. No.: 15/082,395

(22) Filed: Mar. 28, 2016

Related U.S. Application Data
(63) Continuation-in-part of application No. 14/234,721, filed on Jan. 24, 2014, now abandoned.

(30) Foreign Application Priority Data
Jul. 28, 2011 (KR) 10-2011-0074856
Jun. 18, 2012 (KR) 10-2012-0064865

(51) Int. Cl. A61B 17/34

(52) U.S. Cl. CPC A61B 17/3468; A61B 2017/00752

(57) ABSTRACT

Provided is a hair transplanter having high precision in hair transplantation which may finely adjust the length of a hair transplantation needle unit, into which a hair root is inserted, according to thicknesses of skin tissue layers within the scalps of individuals during hair transplantation. The hair transplanter includes a central rod including a guide hole, a hair transplantation needle unit inserted into the guide hole of the central rod, a central case body including a conical cover and a waist part having a small diameter, formed at the front end thereof, and a fixing cover and a tuning cover, formed at the rear end thereof, a core shaft configured to push a hair to be transplanted, which is inserted into the hair transplantation needle unit, and a pressing button including an adjustment lever to fix the core shaft and a lock nut to fix the adjustment lever.

### BACKGROUND OF THE INVENTION

1. Field of the Invention

The present invention relates to a hair transplanter to transplant hair to a bald part, and more particularly to a hair transplanter which may finely adjust the length of a hair transplantation needle unit, into which a hair root is inserted, according to thicknesses of skin tissue layers within the scalps of individuals during hair transplantation.

2. Description of the Related Art

A hair transplanter is an apparatus used to transplant natural hair or artificial hair to a part of the skin of the human body where hair or body hair is lost due to disease or accidents. In general, the hair transplanter is used to transplant eyebrows, beard hair, pubic hair and hair. In the case of a single hair transplantation method which has been conventionally used, individual hair roots are separated from the human body one by one and then transplanted into a desired region using a needle of a hair transplantation needle unit, bleeding and scars may be prevented and the direction, angle and density of hair may be arbitrarily adjusted and, thus, the shape of hair after hair transplantation is naturally maintained and the conventional single hair transplantation method has been widely used in transplantation of eyelashes, beard hair, eyebrows and pubic hair as well as hair. In the case of conventional hair transplanters, when a hair root connected to a hair is located on the inner front surface of a hair transplantation needle unit of the hair transplanter and then the hair transplantation needle unit is inserted into the scalp, the hair transplantation needle unit forms a hole and the hair root is received within the scalp along the hair transplantation needle unit. At this time, in order to push the hair root into skin tissue within the scalp, a core shaft, which moves within the hair transplantation needle unit so as to be slidable, may be used, or a guide plate, which serves as a core shaft and protrudes from the front surface of a sliding member, may be used so that the hair transplantation needle unit is retracted and is not moved forwards any more by the guide plate.

In most conventional hair transplanters, the length of a hair transplantation needle unit is fixed when the hair transplanters are initially manufactured and sold. If hair roots are transplanted into the scalp, since skin tissues within the scalps of individuals have different thicknesses, an operator performs hair transplantation while adjusting the length of the hair transplantation needle unit inserted into the scalp depending on experience and may thus cause damage to the skin tissue within the scalp and generate excessive bleeding or inflammation. Recently, a hair transplanter in which the length of a hair transplantation needle unit may be adjusted has been developed, but is not widely used now due to the complicated structure and high price thereof.

In conventional hair transplanters, since scalp layers of individuals differ according to their scalps, it is difficult for an operator to finely adjust the length of a hair transplantation needle unit of the hair transplanter according to the positions of the tops of the heads of individuals while performing transplantation, and the operator needs to adjust the length of the hair transplantation needle unit one by one and thus a long setting time is taken.

### SUMMARY OF THE INVENTION

Therefore, the present invention has been made in view of the above problems, and it is an object of the present invention to provide a hair transplanter which may finely adjust the length of a hair transplantation needle unit, into which a hair root is inserted, according to thicknesses of skin tissue layers within the scalps of individuals during hair transplantation so as to prevent damage to skin tissue in the scalp, to stably position the hair root within the scalp, and to push the hair root to a desired depth within the scalp to shorten a time taken to perform hair transplantation.

It is another object of the present invention to provide a hair transplanter having high precision in hair transplantation which may finely adjust the lengths of a hair transplantation needle unit and a core shaft, as needed, and have a locking function so as to maintain the finely adjusted state of the hair transplantation needle unit and the core shaft during hair transplantation.

In accordance with an aspect of the present invention, the above and other objects can be accomplished by the provision of a hair transplanter having high precision in hair transplantation including a central rod including a body having a cylindrical shape and provided with a needle support formed at the front part thereof so that a needle enters and exits the body therethrough and a guide hole formed on the side surface thereof, and an upper protrusion having a strew thread and protruding from the rear end of the body, a hair transplantation needle unit inserted into the guide hole and including a needle provided with a hair fixing groove so as to mount a hair to be transplanted therein, a needle head, and protrusions, a central case body having a hollow formed therein in the length direction so that the central rod is inserted into the hollow, the diameter of the hollow at a waist part of the central case body being smaller than that of other parts, the central case body including a conical cover combined with the front end thereof and a screw thread formed at the rear end thereof so as to be combined with a fixing cover and a tuning cover, a core shaft combined with the rear part of the hair transplantation needle unit so as to push the hair to be transplanted, inserted into the hair fixing groove of the needle of the hair transplantation needle unit, out of the hair transplantation needle unit, and including a spring member mounted between the hair transplantation needle unit and the core shaft so as to restore the hair transplantation needle unit to an original position thereof, and a pressing button mounted at the rear part of the core shaft so as to finely adjust a movement range of the core shaft within the needle of the hair transplantation needle unit, wherein the pressing button includes an adjustment lever and a lock nut provided in front of the adjustment lever to lock the adjustment lever.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of a hair transplanter having high precision in hair transplantation in accordance with one embodiment of the present invention;

FIG. 2 is an exploded perspective view of the hair transplanter having high precision in hair transplantation in accordance with the embodiment of the present invention;

FIG. 3 is a partially assembled view of the hair transplanter having high precision in hair transplantation in accordance with the embodiment of the present invention;

FIG. 4 is a cross-sectional view of the hair transplanter having high precision in hair transplantation in accordance with the embodiment of the present invention;

FIGS. 5A and 5B are cross-sectional views illustrating an operating state of the hair transplanter having high precision in hair transplantation in accordance with the embodiment of the present invention; and

FIG. 6 is a cross-sectional view of a hair transplanter having high precision in hair transplantation in accordance with another embodiment of the present invention.

### DETAILED DESCRIPTION OF THE INVENTION

Now, a hair transplanter having high precision in hair transplantation in accordance with a preferred embodiment of the present invention will be described in detail with reference to the annexed drawings.

Terms used in the following description or claims are defined taking into consideration the functions obtained in accordance with the present invention, and the definitions of these terms should be determined based on the whole content of this specification because they may be changed in accordance with the option of a user or usual practice.

Therefore, the description of elements stated in the embodiments and illustrated in the drawings has been made only for better understanding of the present invention.

Those skilled in the art will appreciate that various modifications, additions, and substitutions to the specific elements are possible, without departing from the scope and spirit of the invention as disclosed in the accompanying claims.

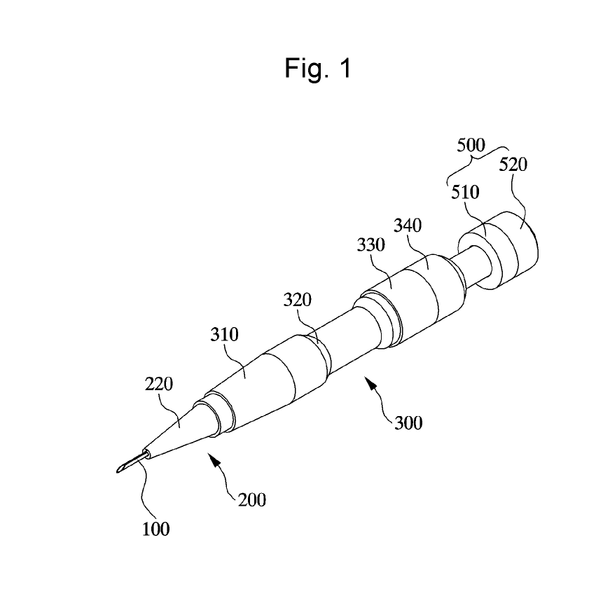

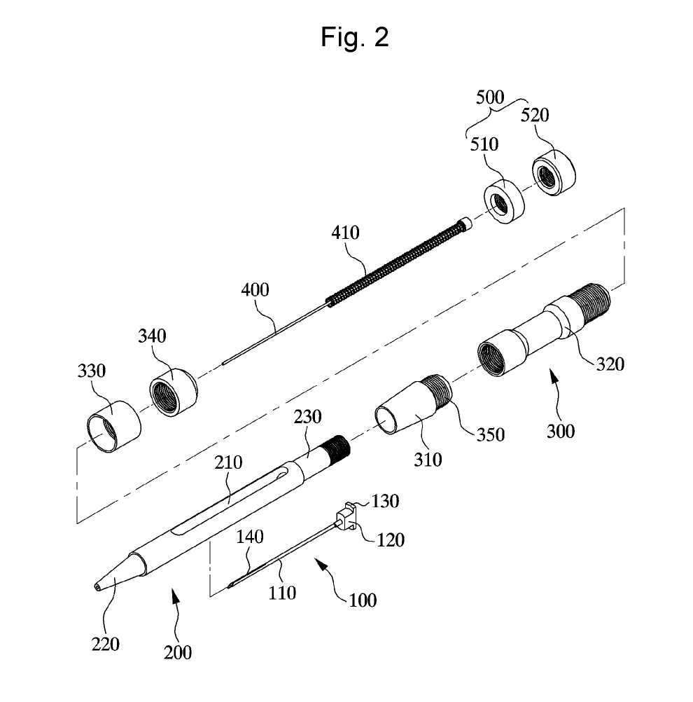

As exemplarily shown in FIGS. 1 to 5B, a hair transplanter having high precision in hair transplantation in accordance with one embodiment of the present invention includes a central rod 200 having a guide hole 210 formed thereon so as to move a hair transplantation needle unit in the forward and backward directions, the hair transplantation needle unit 100 inserted into the guide hole 210 of the central rod 200, a central case body 300 configured to fix the hair transplantation needle unit 100 and including a conical cover 310 and a waist part 320 having a small diameter, formed at the front end thereof, and a fixing cover 330 and a tuning cover 340, formed at the rear end thereof, a core shaft 400 combined with the hair transplantation needle unit 100 so as to push a hair to be transplanted, which is inserted into the hair transplantation needle unit 100, and a pressing button 500 including an adjustment lever 520 to fix the core shaft 400 and a lock nut 510 to fix the adjustment lever 520.

The present invention relates to a hair transplanter which may increase precision in hair transplantation.

Hereinafter, the above-described configuration of the hair transplanter having high precision in hair transplantation in accordance with the present invention will be described in more detail.

As exemplarily shown in FIGS. 1 to 5B, the central rod 200 of the of the hair transplanter having high precision in hair transplantation of the present invention includes a body having a cylindrical shape so as to mount the hair transplantation needle unit 100 therein, a needle support 220 formed at the front part of the body and having a through hole through which a needle 110 enters and exits the central rod 200, and the guide hole 210 formed on the side surface of the body. An upper protrusion 230 having a screw thread is formed at the rear end of the body of the central rod 200, the core shaft 400 is inserted into the upper protrusion 230, and the pressing button 500 is mounted at the upper protrusion 230 so as to prevent removal of the core shaft 400.

The hair transplantation needle unit 100 of the present invention includes a needle head 120 formed at the rear end thereof, protrusions 130 formed on the side surfaces of the needle head 120 so as to move the hair transplantation needle unit 100 within the guide hole 210, and a hair fixing groove 140 formed on the surface of the needle 110 so that the hair to be transplanted may be inserted into the hair fixing groove 140.

Further, a plurality of scale marks is formed on the surface of the needle 110 so that an operator may visually confirm the inserted depth of the needle 110 into the scalp.

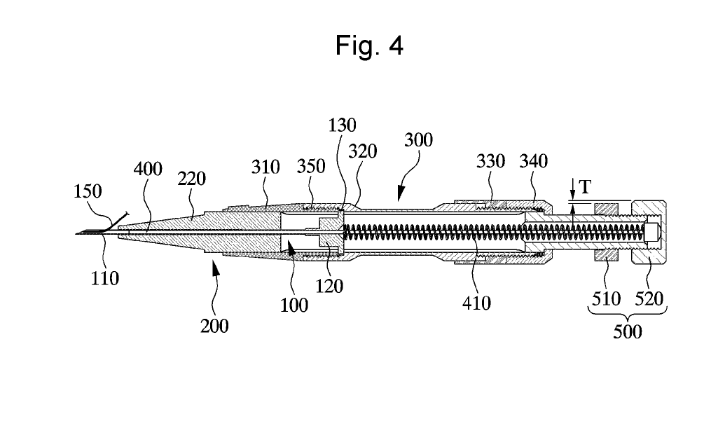

As exemplarily shown in FIG. 4, the core shaft 400 is inserted into the needle head 120 of the hair transplantation needle unit 100 and is inserted into the needle 110 within the scalp so as to push a hair 150 to be transplanted, thereby achieving hair transplantation.

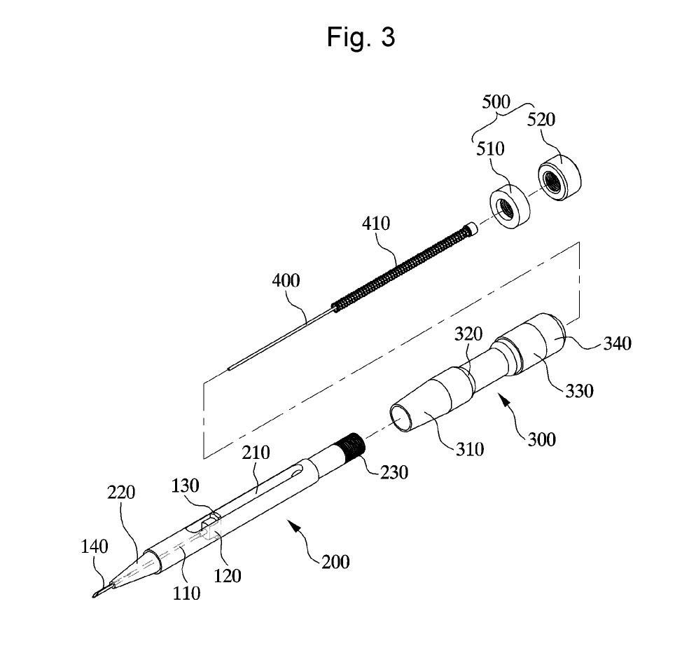

As exemplarily shown in FIG. 3, the central case body 300 has a hollow formed therein in the length direction so that the central rod 200 is inserted into the hollow to be combined with the central case body 300, and the diameter of the hollow at the waist part 320 of the central case body 300 is smaller than that of other parts.

The conical cover 310 is combined with the front part of the central case body 300, and a screw thread is formed at the rear part of the central case body 300 so that the fixing cover 320 and the tuning cover 330 may be combined with the screw thread formed at the rear end of the central case body 300.

Further, the needle head 120 of the hair transplantation needle unit 100 shown in FIG. 4 is inserted into the central case body 300, and the conical cover 310 is tightly coupled with the front part of the central case body 300 such that the protrusions 130 of the needle head 120 are placed on a mount part 350 of the conical cover 310 and fixed closely to the waist part 320 of the central case body 300.

That is, the protrusions 130 of the needle head 120 are fixed closely between the conical cover 310 and the waist part 320 of the central case body 300.

The core shaft 400 of the present invention is inserted into the hair transplantation needle unit 100 so as to push the hair to be transplanted 150, which is inserted into the hair fixing groove 140 formed on the surface of the needle 110, within the hair transplantation needle unit 100, and a spring member 410 is mounted between the needle head 120 of the hair transplantation needle unit 100 and the core shaft 400 so as to restore the hair transplantation needle unit 100 to an original position thereof.

Further, in the present invention, the adjustment lever 520 is located at the rear of the central case body 300 and combined with the rear part of the central rod 200 so as to apply pressure to the core shaft 400 to achieve fine adjustment of a movement range (d) of the core shaft 40 within the needle 110, and the lock nut 510 is formed in front of the adjustment lever 520 so as to fix the adjustment lever 520. In the hair transplanter having high precision in hair transplantation in accordance with the embodiment of the present invention, an operator conveniently fixes the adjustment lever 520 using the lock nut 510 so as not to be loosened.

The lock nut 510 is formed in front of the adjustment lever 520 so as to maintain and fix the finely adjusted state of the adjustment lever 520, and there is a height difference T between the adjustment lever 520 and the lock nut 510 so as to conveniently adjust the adjustment lever 520.

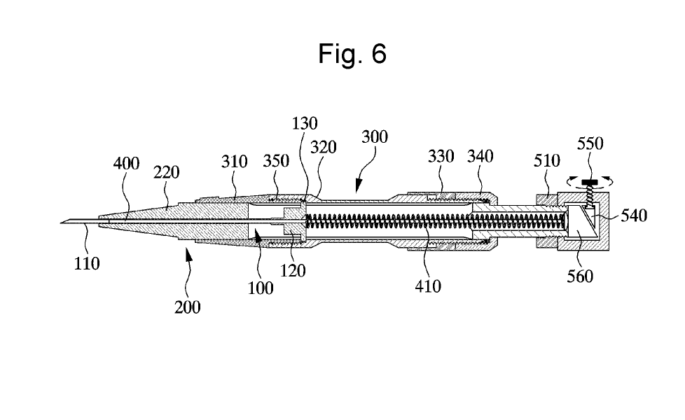

As exemplarily shown in FIG. 6, an adjustment lever 520 in accordance with another embodiment of the present invention includes an upper part 540, a lower part 560, and a lever bolt 550 to move the upper part 540 in the forward and backward directions, and thus adjusts the movement range of the core shaft 400 within the needle 110.

The lever bolt 550 is formed on the side surface of the adjustment lever 520, and the upper part 540 having an inverted triangular structure and connected to the lever bolt 550 and the lower part 560 having an inclined surface contacting the upper part 540 are formed within the adjustment lever 510. When the lever bolt 440 is rotated in a regular direction, the upper part 540 moves downwards along the inclined surface of the lower part 560 and thus the lower part 560 moves backwards. On the other hand, when the lever bolt 550 is rotated in the opposite direction, the upper part 540 moves upwards along the inclined surface of the lower part 560 and thus the lower part 560 moves forwards by the spring member 410 mounted in the core shaft 400. That is, when the lever bolt 550 is rotated in the above-described manner, the movement range of the core shaft 400 may be finely adjusted.

Further, in the present invention, scale marks are formed on the needle 110 of the hair transplantation needle unit 110 so that an operator may visually confirm the inserted depth of the needle 110 into the scalp.

The hair transplanter having high precision in hair transplantation in accordance with the present invention will be described in detail with reference to the accompanying drawings.

First, conventional hair transplanters and hair transplantation using a slit method respectively have advantages and disadvantages. As compared to hair transplantation using the slit method, the conventional hair transplanters are disadvantageous in that it is difficult to adjust the transplantation depth of a hair.

The hair transplanter in accordance with the present invention allows an operator to transplant a hair to a desired position through adjustment of the lengths of the hair transplantation needle unit and the core shaft so as to increase the survival rate of the transplanted hair based on a theory about the optimized depth of a transplanted hair.

First, as exemplarily shown in FIG. 4, after preparation for hair transplantation by inserting a hair to be transplanted 150 into the hair fixing groove 140 of the needle 110, hair transplantation on the scalp is carried out.

Here, since the thicknesses of the scalps of individuals are different, an operator judges the state of the scalp of a patient and performs hair transplantation so that the hair to be transplanted 150 is safely positioned at a correct position within the scalp.

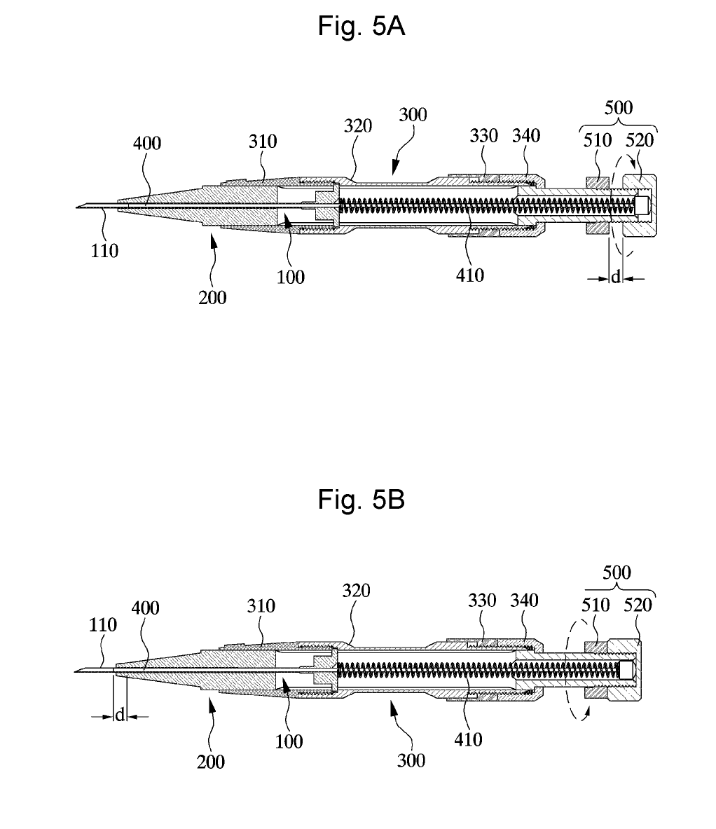

In the present invention, as exemplarily shown in FIGS. 5A and 5B, in order to allow an operator to transplant a hair to be transplanted to a desired position to increase the survival rate of the transplanted hair, the adjustment lever 520 is provided to finely adjust the length of the core shaft 400 which pushes the hair to be transplanted within the needle 100 during hair transplantation, and the lock nut 510 is rotated to maintain the finely adjusted state of the adjustment lever 520 without change of the length of the core shaft 400 due to loosening of the adjustment lever 520 after adjustment of the length of the core shaft 400 using the adjustment lever 520.

The adjustment lever 520 and the lock nut 510 form the pressing button 500 mounted at the rear part of the core shaft 400 so as to apply pressure to the core shaft 400.

When the operator presses the pressing button 500 with his/her thumb, the core shaft 400 move forwards and the hair to be transplanted, which is inserted into the needle 110, is pushed out.

The above-described adjustment of the core shaft 400 is fine adjustment of the movement range (d) of the core shaft 400 within the needle 110 and, thereby, the position of the hair to be transplanted within the scalp may be finely adjusted so as to increase the survival rate of the transplanted hair.

Therefore, when the adjustment lever 520 is loosened, the core shaft 400 may move backwards within the needle 110 and, when the adjustment lever 520 is tightened, the core shaft 400 may move forwards within the needle 110 so as to transplant a hair deeply into the scalp.

Here, fine adjustment of the operating length of the core shaft 400 is carried out by rotating the adjustment lever 520, and the lock nut 520 formed in front of the adjustment lever 520 is rotated to fix the adjustment lever 520 without rotation of the adjustment lever 520 so that the length of the core shaft 400 at the adjusted position is not changed.

The hair transplanter in accordance with the present invention may conveniently achieve fine adjustment of the length of the core shaft 400 when a hair is transplanted into the scalp, and then fix the length of the core shaft 400 using the lock nut 520 after fine adjustment of the length of the core shaft 400 during hair transplantation.

As apparent from the above description, a hair transplanter in accordance with the present invention may conveniently achieve fine adjustment of the length of a hair transplantation needle unit, into which a hair root is inserted, according to thicknesses of skin tissue layers within the scalps of individuals during hair transplantation. Further, the hair transplanter in accordance with the present invention may have a locking function of fixing the hair transplantation needle unit in the finely adjusted state, thereby causing hair to be transplanted regularly and allowing an operator to conveniently execute hair transplantation.

Moreover, the hair transplanter in accordance with the present invention may form scale marks on the surface of a needle of the hair transplantation needle unit so that an operator may visually confirm the inserted depth of the needle into the scalp and, particularly, fix the hair transplantation needle unit moved to a fine adjustment section.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A hair transplanter having high precision in hair transplantation comprising:
a central rod including a body having a cylindrical shape and provided with a needle support formed at the front part thereof so that a needle enters and exits the body therethrough and a guide hole formed on the side surface thereof, and an upper protrusion having a strew thread and protruding from the rear end of the body;
a hair transplantation needle unit inserted into the guide hole and including a needle provided with a hair fixing groove so as to mount a hair to be transplanted therein, a needle head, and protrusions;
a central case body having a hollow formed therein in the length direction so that the central rod is inserted into the hollow, the diameter of the hollow at a waist part of the central case body being smaller than that of other parts, the central case body including a conical cover combined with the front end thereof and a screw thread formed at the rear end thereof so as to be combined with a fixing cover and a tuning cover;
a core shaft combined with the rear part of the hair transplantation needle unit so as to push the hair to be transplanted, inserted into the hair fixing groove of the needle of the hair transplantation needle unit, out of the hair transplantation needle unit, and including a spring member mounted between the hair transplantation needle unit and the core shaft so as to restore the hair transplantation needle unit to an original position thereof; and
a pressing button mounted at the rear part of the core shaft so as to finely adjust a movement range of the core shaft within the needle of the hair transplantation needle unit,
wherein the pressing button includes an adjustment lever and a lock nut provided in front of the adjustment lever to lock the adjustment lever.

2. The hair transplanter having high precision in hair transplantation according to claim 1, wherein the lock nut is provided in front of the adjustment lever to fixedly maintain a finely adjusted state of the adjustment lever, and the diameter of the lock nut is greater than the diameter of the adjustment lever so as to conveniently achieve adjustment.

3. The hair transplanter having high precision in hair transplantation according to claim 1, wherein:
the adjustment lever includes a lever bolt formed on the side surface thereof, an upper part having an inverted triangular structure and formed at the end of the lever bolt, and a lower part having an inclined surface contacting the upper part; and
the adjustment lever is configured such that, when the lever bolt is rotated in a regular direction, the upper part moves downwards along the inclined surface and thus the lower part moves backwards and, when the lever bolt is rotated in the opposite direction, the upper part moves upwards along the inclined surface and thus the lower part moves forwards by the spring member mounted in the core shaft.

4. The hair transplanter having high precision in hair transplantation according to claim 1, wherein scale marks are formed on the needle of the hair transplantation needle unit.
